(19) United States
(12) Patent Application Publication
(10) Pub. No.: US 2009/0030189 A1
(43) Pub. Date: Jan. 29, 2009

(54) mTOR GLS and ELS, their small molecule mimetics and competitive inhibitors

(76) Inventors: X. F. Steven Zheng, Watchung, NJ (US); Xiangyu Liu, Piscataway, NJ (US)

Correspondence Address: University of Medicine and Dentistry of New Jersey;Office of Patents and Licensing, Suite 2100, One Worlds Fair Drive, Somerset, NJ, 08873, US

(21) Appl. No.: 12/001,161

(22) Filed: Dec. 10, 2007

Related U.S. Application Data
(60) Provisional application No. 60/873,761, filed on Dec. 8, 2006.

(51) Int. Cl. C07H 21/04

(52) U.S. Cl. 536/23.1

(57) ABSTRACT

The present invention relates generally to molecular mechanisms of mTOR-related human diseases. More specifically, the invention relates to two novel related polypeptides, the ER-localization sequence (ELS) and Golgi-localization sequence (GLS) along with therapeutic, diagnostic and research utilities for these polynucleotides and proteins.

### BACKGROUND OF THE INVENTION

1. Field of the Invention

The present invention relates generally to molecular mechanisms of mTOR-related human diseases. More specifically, the invention relates to two novel related polypeptides, the ER-localization sequence (ELS) and Golgi-localization sequence (GLS) along with therapeutic, diagnostic and research utilities for these polynucleotides and proteins.

2. Related Art

Rapamycin and analogs are promising anticancer agents currently under clinical trials (Huang and Houghton, 2003). When complexed with FKBP12, rapamycin binds to and inhibits the target of rapamycin (TOR) protein, a 289 kDa phosphatidylinositide 3-kinase (PI3K)-related kinase (PIKK). TOR proteins are well conserved in all eukaryotes, with over 45% amino acid sequence identity and 70% similarity between yeast and human TOR proteins. The C-terminal PIKK kinase domain has protein serine and threonine kinase activity (Hunter, 1995; Keith and Schreiber, 1995) essential for TOR functions (Brown et al., 1995; Zheng et al., 1995). The PIKK and two other C-terminal domains, FAT and FATC, form the signature for the PIKK family that also includes ATM, ATR and DNA-PK (Bossini et al., 2000). FRB (FKBP12-rapamycin binding) domain is a conserved 11 kDa region necessary and sufficient for FKBP12-rapamycin binding (Chen et al., 1995; Zheng et al., 1995). A conserved serine residue in FRB domain, Ser-2035 in mTOR and Ser-1972 in yeast Tor1, is crucial for the binding of FKBP12-rapamycin. When mutated to a bulkier residue such as threonine, it disrupts the binding of FKBP12-rapamycin and confers dominant-rapamycin resistance (Brown et al., 1995; Zheng et al., 1995). The N-terminal half of TOR contains 20 tandem repeats of 37-43 amino acids termed HEAT (HT) repeats (Andrade and Bork, 1995). A single HEAT repeat forms a pair of interacting anti-parallel helices linked by a flexible intra-unit loop. HEAT repeats occur in tandem clusters, linked by flexible inter-unit spacers (IUS). Crystal structures of HEAT repeat proteins such as the A subunit of phosphatase 2A show that HEAT repeats form superhelical scaffoldings, which engage in interaction with other proteins (Groves et al., 1999). Indeed, the HEAT repeats of TOR proteins have been shown to mediate interaction with proteins such as Gln3 (Bertram et al., 2000) and Kog1/Raptor (Kim et al., 2002).

TOR forms two distinct protein complexes, TOR complexes 1 and 2 (TORC1 and TORC2). The major components of mammalian TORC1 (mTORC1) are mTOR, Raptor/mKog1 and GβL/mLst8 (Hara et al., 2002; Kim et al., 2002; Loewith et al., 2002; Kim et al., 2003), and those of mTORC2 include mTOR, mAvo1, Rictor/mAvo3 and GβL/mLst8 (Jacinto et al., 2004; Sarbassov et al., 2004). While TORC1 is sensitive to rapamycin, TORC2 is not (Loewith et al., 2002), which is in agreement with two previously defined functions for TOR in yeast (Zheng et al., 1995). mTORC1 integrates signals from growth factors and nutrients, and regulates diverse growth-related processes, including translational initiation, ribosome biogenesis and autophagy. Translational initiation is the best understood TORC1-dependent process. mTORC1 phoshorylates several translational regulators, including ribosomal S6 kinases (S6Ks) and eIF4E-binding protein 1 (4E-BP1, also called PHAS-I)(Dennis et al., 1999; Inoki and Guan, 2006; Kuruvilla and Schreiber, 1999; Schmelzle and Hall, 2000; Raught et al., 2001; Rohde et al., 2001; McDaniel et al., 2002). The functions of mTORC2 are much less well studied. mTORC1 has been shown to be required for actin organization (Jacinto et al., 2004; Sarbassov et al., 2004).

The endoplasmic reticulum (ER) and the Golgi apparatus are part of the secretory pathway engaging in synthesis, modification and transport of secreted and plasma membrane proteins (Baumann and Walz, 2001). The ER and the Golgi also participate in intracellular signaling and other aspects of cell regulation, including the unfolded protein response (UPR) and the ER overload response (EOR) (Pahl, 1999). More recently, several studies demonstrate that the ER is involved in signal transduction pathways traditionally thought to emanate from the plasma membrane. For example, the small GTPase Ras restricted to the Golgi apparatus can actively engage signal transduction to MAP kinases (Chiu et al., 2002). How the ER and the Golgi anchor intracellular signaling is elegantly illustrated by the sterol sensing and signaling pathway involving sterol regulatory element-binding proteins (SREBPs) (Rawson, 2003). When cholesterol level is high, SCAP binds to cholesterol in the ER membrane and assumes a conformation that promotes binding to the ER-resident protein INSIG (Brown et al., 2002; Yang et al., 2002). This retains the SREBP-SCAP complex in the ER by preventing interaction of SCAP with COPII vesicle-formation proteins Sar1, Sec23 and Sec24. When cholesterol level is low, SREBP-SCAP is dissociated from INSIG and is transported by COPII-coated vesicles to the Golgi, where SREBP is sequentially cleaved by two proteases, S1P and S2P, leading to the release of the N-terminal transcriptional activation domain from the Golgi membrane to the nucleus, where it activates the target genes (Wang et al., 1994; Sakai et al., 1996).

Differential signaling output is fundamentally dependent on the spatial organization of the signaling molecules, their regulators and effectors within the cell. How signaling molecules are targeted to different subcellular compartments is an important but still poorly understood question for signal transduction research. Despite the central role of mTOR in cell growth and functions, relatively little is known about its precise subcellular distribution, and to an even lesser degree the underlying mechanisms and functional significance. We have previously shown that mTOR is localized to the ER and Golgi in several common mammalian cell lines (Drenan et al., 2004).

### SUMMARY OF THE INVENTION

The present invention is drawn to GLS and ELS polypeptides, as well as DNA segments encoding GLS and ELS polypeptides and antibodies. The present invention also provides methods of making such DNA segments and polypeptides, as well as their use as research tools, in drug screening, diagnosis of various clinical conditions associated with mTor, immunosuppressive and cancer therapy. Cells relating to GLS and ELS are disclosed as well.

Thus, in a particular aspect of the invention, there is provided a purified or a substantially purified ELS—SEQ ID NO:1 (FIG. 9) or GLS SEQ ID NO:2 (FIG. 9) protein or polypeptide. Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition. In certain embodiments, the protein or polypeptide of the invention may be operatively linked to a second polypeptide sequence.

In another embodiment, an isolated nucleic acid segment encoding a polypeptide comprising the sequence as shown in SEQ ID NO:1 and SEQ ID NO:2. is provided. The nucleic acid segment may comprise the DNA sequence as shown in SEQ ID NO:1. and SEQ ID NO:2. The nucleic acid segment may further comprise a promoter operably linked to the region encoding the protein. The promoter may be an inducible promoter, a constitutive promoter or a tissue specific promoter. The nucleic acid segment may be comprised within a viral vector, such as an adenoviral vector, a retroviral vector, an adeno-associated viral vector, a vaccinia viral vector, a herpesviral vector or a pox viral vector. The nucleic acid segment may be comprised within a non-viral vector. The non-viral vector may be comprised in a lipid carrier. The nucleic acid segment may further comprise a region encoding a selectable marker protein.

Examples of constitutive viral promoters include the HSV, TK, RSV, LTR promoter sequence from retroviral vectors, SV40 and CMV promoters, of which the CMV promoter is a currently preferred example. Examples of constitutive mammalian promoters include various housekeeping gene promoters, as exemplified by the .beta. actin promoter. Other promoters may be dectin-1, dectin-2, human CD11c, F4/80, SM22, RSV, SV40, Ad MLP, .beta.-actin, MHC class I or MHC class II promoter,

Inducible promoters and/or regulatory elements are also contemplated for use with the expression vectors of the invention. Examples of suitable inducible promoters include promoters from genes such as cytochrome P450 genes, heat shock protein genes, metallothionein genes, hormone-inducible genes, such as the estrogen gene promoter, and such like. Promoters that are activated in response to exposure to ionizing radiation, such as fos, jun and egr-1, are also contemplated.

The nucleic acid segment also may be characterized as (a) a nucleic acid segment comprising a sequence region that consists of 14 nucleotides that have the same sequence as, or complementary to, at least 14 contiguous nucleotides of SEQ ID NO:1; SEQ ID NO:2 or (b) a nucleic acid segment of from 14 to 10,000 nucleotides in length that hybridizes to the nucleic acid segment of SEQ ID NO:1, SEQ ID NO:2 or the complement thereof, under stringent hybridization conditions. The segment may comprise a sequence region of at least 14, 17, 20, 25 or 30 contiguous nucleotides from SEQ ID NO:1 or the complement thereof. The segment may be 17, 20, 25 or 30 nucleotides in length.

Nucleic acids of the invention may also be operatively linked to other protein-encoding nucleic acid sequences. This will generally result in the production of a fusion protein following expression of such a nucleic acid construct. Both N-terminal and C-terminal fusion proteins are contemplated. Virtually any protein- or polypeptide-encoding DNA sequence, or combinations thereof, may be fused to an ELS or GLS sequence in order to encode a fusion protein. This includes DNA sequences that encode targeting polypeptides, therapeutic proteins, proteins for recombinant expression, proteins to which one or more targeting polypeptides is attached, protein subunits and the like.

Vectors and plasmids may be constructed with at least one multiple cloning site. In certain embodiments, the expression vector will comprise a multiple cloning site that is operatively positioned between a promoter and an ELS or GLS gene sequence. Such vectors may be used, in addition to their uses in other embodiments, to create N-terminal fusion proteins by cloning a second protein-encoding DNA segment into the multiple cloning site so that it is contiguous and in-frame with the ELS or GLS sequence.

In other embodiments, expression vectors may comprise a multiple cloning site that is operatively positioned downstream from the expressible ELS or GLS sequence. These vectors are useful, in addition to other uses, in creating C-terminal fusion proteins by cloning a second protein-encoding DNA segment into the multiple cloning site so that it is contiguous and in-frame with the ELS or GLS sequence. Vectors and plasmids in which a second protein- or RNA-encoding nucleic acid segment is also present are, of course, also encompassed by the invention, irrespective of the nature of the nucleic acid segment itself. Expression vectors may also contain other nucleic acid sequences, such as IRES elements, polyadenylation signals, splice donor/splice acceptor signals, and the like.

Particular examples of suitable expression vectors are those adapted for expression using a recombinant adenoviral, recombinant adeno-associated viral (AAV) or recombinant retroviral system. Vaccinia virus, herpes simplex virus, cytomegalovirus, and defective hepatitis B viruses, amongst others, may also be used.

Recombinant host cells form another aspect of the present invention. Such host cells will generally comprise at least one copy of an isolated ELS or GLS sequence linked to a heterologous promoter. Preferred cells for expression purposes will be prokaryotic host cells or eukaryotic host cells. Accordingly, cells such as bacterial, yeast, fungal, insect, nematode and plant cells are also possible. An example of a preferred bacterial host cell is E. coli. Examples of suitable eukaryotic host cells include VERO cells, HeLa cells, cells of Chinese hamster ovary (CHO) cell lines, COS cells, such as COS-7, and W138, BHK, HepG2, 3T3, RIN, MDCK, A549, PC12, K562 and 293 cells. Cells also include transgenic cells derived from transgenic animals engineered to overexpress or not express ELS or GLS sequence, or to express a screenable marker under the control of ELS or GLS sequence regulatory signals. The marker may be luciferase, green fluorescent protein or any other gene whose expression is readily detected.

Many methods of using ELS or GLS sequences are obtained from the present invention, such as expressing an ELS or GLS protein in a cell.

GLS and ELS expression and/or activity may be used to treat a number of for many human diseases, including graft rejection, autoimmunity, restenosis, cancer, heart diseases, diabetes, obesity, aging, and Alzheimer's, Parkinson's and Huntington's diseases. The inventors contemplate that ELS and GLS proteins and ELS and GLS expression constructs will inhibit mTORC1 and mTORC2 activity, expression and/or activity.

ELS or GLS or an immunologically active fragment thereof, may be used to inoculate an animal in order to produce polyclonal antibodies which react with ELS or GLS. By “immunologically active fragment” is meant a fragment of the approximately ELS or GLS protein which fragment is sufficient to stimulate production of antibodies which specifically react with an exposed epitope. Thus, in addition to ELS or GLS, the present invention contemplates other antibodies, polyclonal or monoclonal, which specifically react with ELS or GLS or an immunologically active fragment thereof.

A protein of the present invention (from whatever source derived, including without limitation from recombinant and non-recombinant sources) may be used in a pharmaceutical composition when combined with a pharmaceutically acceptable carrier. Such a composition may also contain (in addition to protein and a carrier) diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration. The pharmaceutical composition may further contain other agents which either enhance the activity of the protein or compliment its activity or use in treatment. Such additional factors and/or agents may be included in the pharmaceutical composition to produce a synergistic effect with protein of the invention, or to minimize side effects.

A protein of the present invention may be active in multimers (e.g., heterodimers or homodimers) or complexes with itself or other proteins. As a result, pharmaceutical compositions of the invention may comprise a protein of the invention in such multimeric or complexed form.

The pharmaceutical composition of the invention may be in the form of a complex of the protein(s) of present invention along with protein or peptide antigens. The antigen components could also be supplied as purified MHC-peptide complexes alone or with co-stimulatory molecules that can directly signal T cells. Alternatively antibodies able to bind surface immunolgobulin and other molecules on B cells as well as antibodies able to bind the TCR and other molecules on T cells can be combined with the pharmaceutical composition of the invention.

The pharmaceutical composition of the invention may be in the form of a liposome in which protein of the present invention is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323, all of which are incorporated herein by reference.

As used herein, the term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

In practicing the method of treatment or use of the present invention, a therapeutically effective amount of protein of the present invention is administered to a mammal having a condition to be treated. Protein of the present invention may be administered in accordance with the method of the invention either alone or in combination with other therapies such as treatments employing cytokines, or other lymphokines factors.

Administration of protein of the present invention used in the pharmaceutical composition or to practice the method of the present invention can be carried out in a variety of conventional ways, such as oral ingestion, inhalation, topical application or cutaneous, subcutaneous, intraperitoneal, parenteral or intravenous injection. Intravenous administration to the patient is preferred.

When a therapeutically effective amount of protein of the present invention is administered orally, protein of the present invention will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% protein of the present invention, and preferably from about 25 to 90% protein of the present invention. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of protein of the present invention, and preferably from about 1 to 50% protein of the present invention.

When a therapeutically effective amount of protein of the present invention is administered by intravenous, cutaneous or subcutaneous injection, protein of the present invention will be in the form of a pyrogen-free, parenterally acceptable aqueous solution. The preparation of such parenterally acceptable protein solutions, having due regard to pH, isotonicity, stability, and the like, is within the skill in the art. A preferred pharmaceutical composition for intravenous, cutaneous, or subcutaneous injection should contain, in addition to protein of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art. The amount of protein of the present invention in the pharmaceutical composition of the present invention will depend upon the nature and severity of the condition being treated, and on the nature of prior treatments which the patient has undergone. Ultimately, the attending physician will decide the amount of protein of the present invention with which to treat each individual patient. Initially, the attending physician will administer low doses of protein of the present invention and observe the patient's response. Larger doses of protein of the present invention may be administered until the optimal therapeutic effect is obtained for the patient, and at that point the dosage is not increased further. It is contemplated that the various pharmaceutical compositions used to practice the method of the present invention should contain about 0.01 .mu.g to about 100 mg (preferably about 0.1 ng to about 10 mg, more preferably about 0.1 .mu.g to about 1 mg) of protein of the present invention per kg body weight.

The duration of intravenous therapy using the pharmaceutical composition of the present invention will vary, depending on the severity of the disease being treated and the condition and potential idiosyncratic response of each individual patient. It is contemplated that the duration of each application of the protein of the present invention will be in the range of 12 to 24 hours of continuous intravenous administration. Ultimately the attending physician will decide on the appropriate duration of intravenous therapy using the pharmaceutical composition of the present invention.

### BRIEF DESCRIPTION OF THE DRAWINGS

The foregoing features of the invention will be apparent from the following Detailed Description of the Invention, taken in connection with the accompanying drawings, in which:

FIG. 1: Amino acids (aa) 931-1039 of mTOR is sufficient to target EGFP to the Golgi apparatus

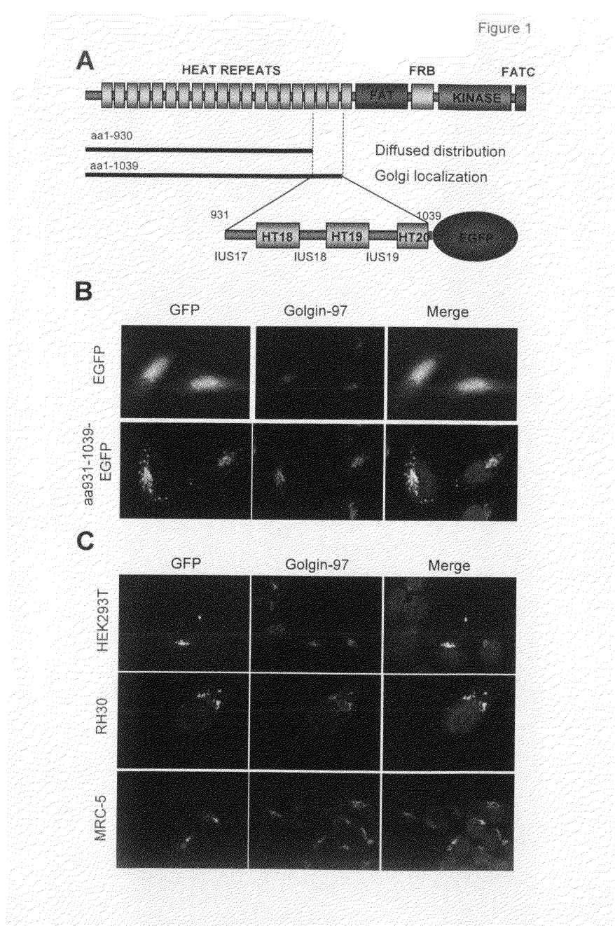

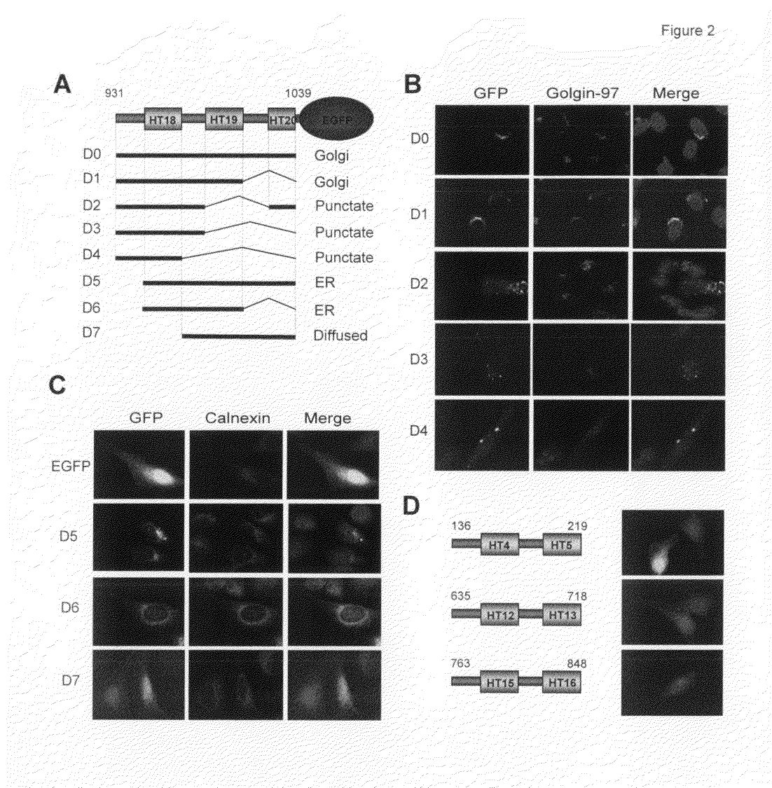

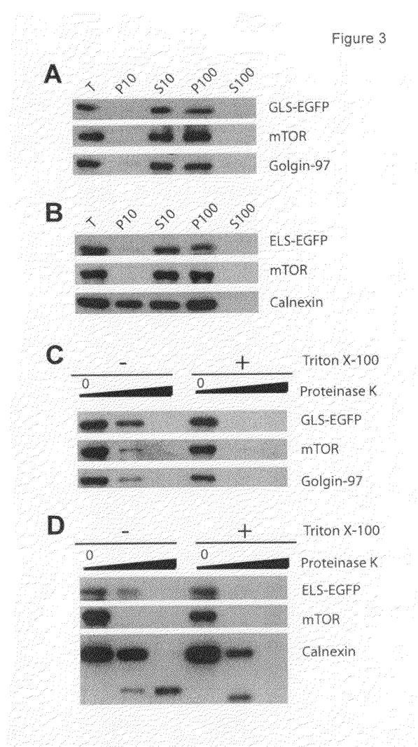

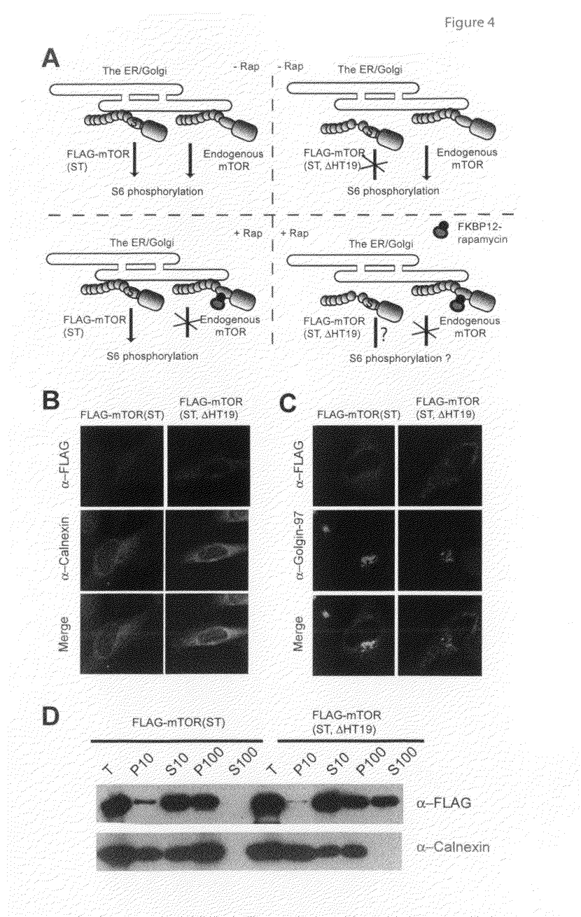

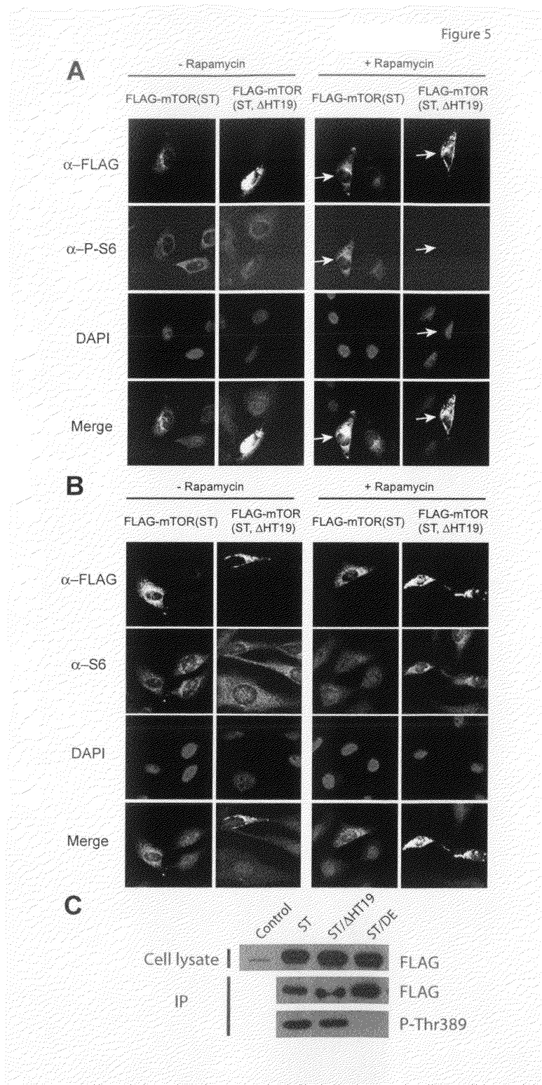

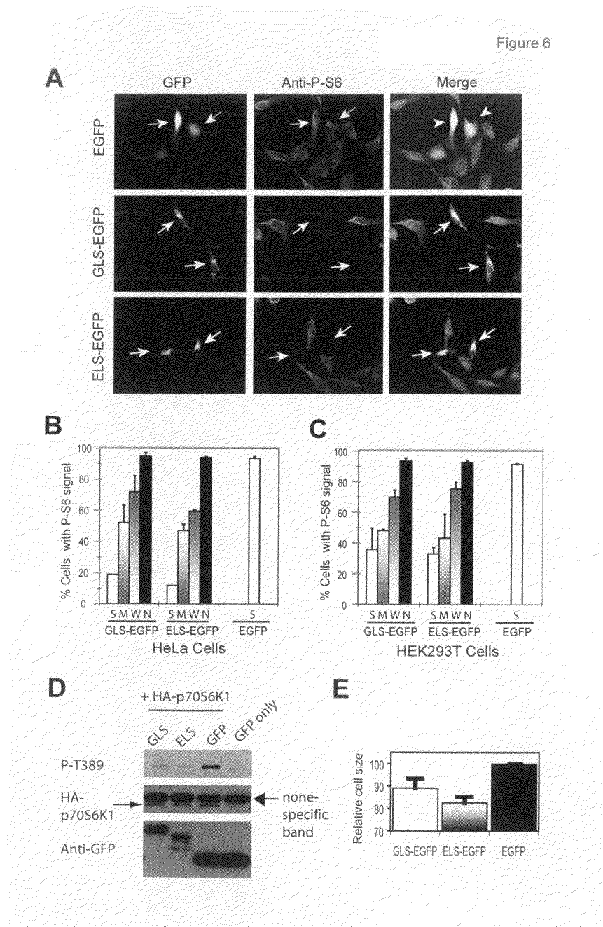

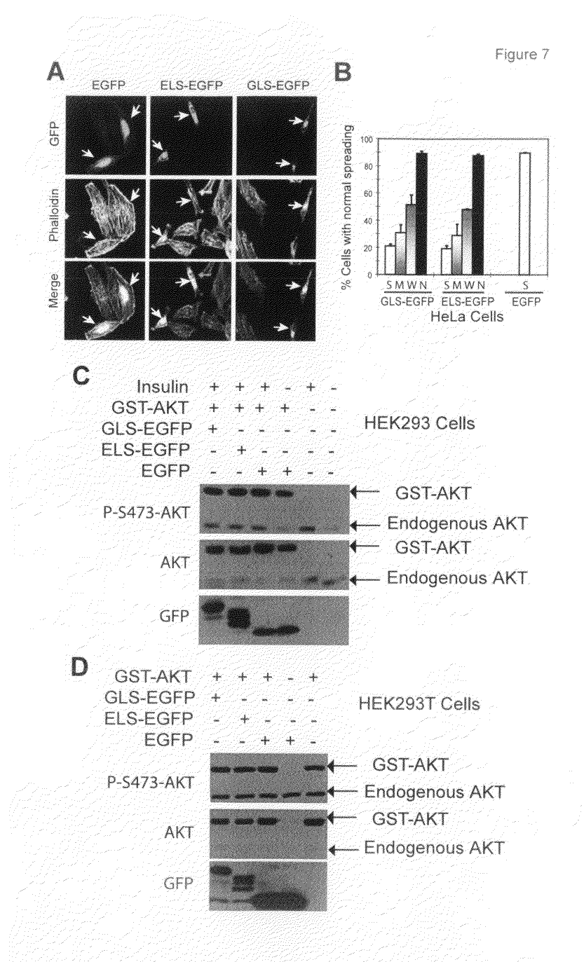

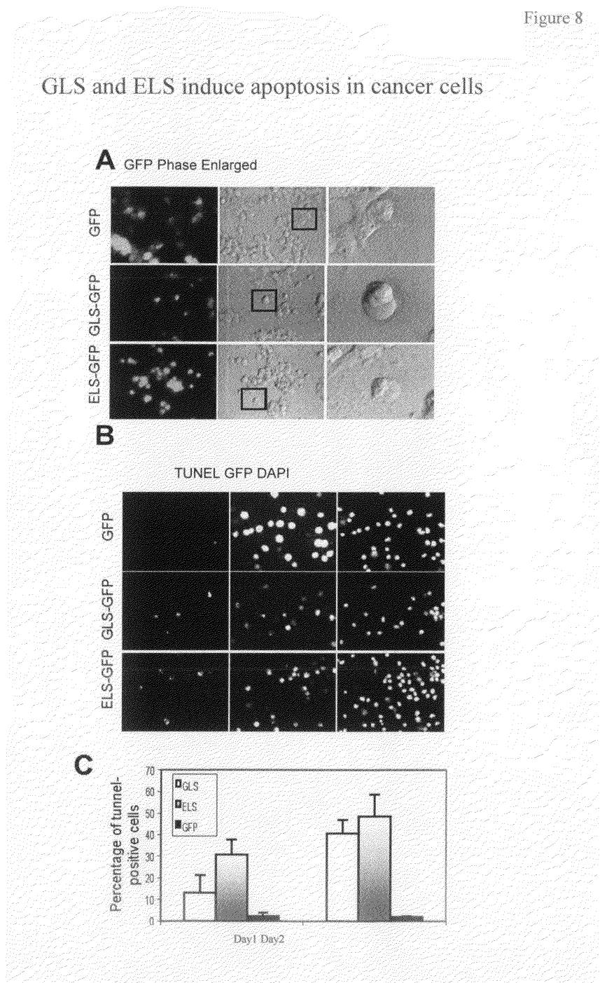

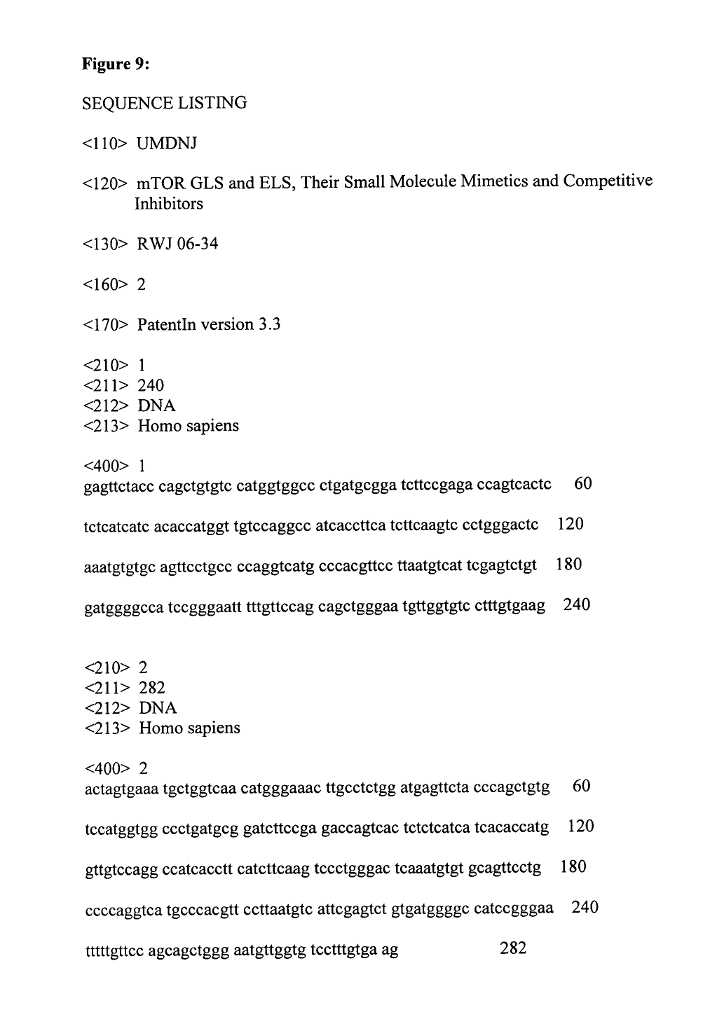

(A) A schematic presentation of mTOR and subcellular localization of two mTOR N-terminal fragments in HeLa cells

(B) aa931-1039-EGFP is predominantly localized to the Golgi in HeLa cells. EGFP or aa931-1039-EGFP is transiently expressed in HeLa cells (Green). The Golgi is visualized by immunofluorescence (IF) staining with a Golgin-97-specific antibody (Red). The nuclei are stained with DAPI (Blue).

(C) aa931-1039-EGFP is localized to the Golgi in several common mammalian cell lines. aa931-1039-EGFP is transiently expressed in HEK293T, RH30 and MRC-5 cells (Green). The Golgi is visualized by IF with a Golgin-97-specific antibody (Red). The nuclei are stained with DAPI (Blue).

FIG. 2: Deletional analysis of the aa931-1039 region reveals that it contains both Golgi- and ER-localization sequences

(A) Summary of the deletional mutants of aa931-1039-EGFP and their subcellular localization

(B) Localization of D1-4 deletional mutants. aa931-1039-EGFP (D0) and D1-4 mutants were transiently expressed in HeLa cells (Green). The Golgi is visualized by IF with a Golgin-97-specific antibody (Red). The nuclei are stained with DAPI (Blue).

(C) Localization of the D5-7 deletional mutants. The D5-7 deletional mutants were transiently expressed in HeLa cells (Green). The ER is visualized by IF with a calnexin-specific antibody (Red). The nuclei are stained with DAPI (Blue). EGFP is used as a control.

(D) ER/Golgi-localization is not a general feature for tandem HEAT repeat pairs. EGFP-fusions with three randomly selected tandem pairs of HEAT repeats and an N-terminal IUS (HT4-5, 12-13, 15-16) were transiently expressed in HeLa cells. Their localizations were analyzed by fluorescence microscopy.

FIG. 3: GLS and ELS share the same topology of ER/Golgi localization as mTOR

(A),(B) Subcellular fractionation of GLS-EGFP and ELS-EGFP. GLS-EGFP and ELS-EGFP were transiently expressed in HEK293T cells. The cells were lysed in hypotonic buffer and the cell lysates were fractionated by differential centrifugation. T, total cell lysates; P10, 10,000 g pellet; S0, 10,000 g supernatant; P100, 100,000 g pellet; S100, 100,000 g supernatant.

(C), (D) GLS-EGFP and ELS-EGFP are localized on the cytoplasmic face of Golgi and ER membranes. The P100 pellets from FIGS. 3A and B were treated with different concentrations of proteinase K (0, 5, 50 μg/ml) in the presence or absence of the detergent Triton X-100. GLS-EGFP and ELS-EGFP were analyzed by Western blot with antibodies specific for GFP.

FIG. 4: Deletion of HT19 causes delocalization of FLAG-mTOR from the ER and Golgi

(A) The strategy for analyzing the importance of HT19 in mTOR localization and function. FLAG-mTOR(S2035T) or FLAG-mTOR(S2035T, ΔHT19) is expressed in HeLa cells. IF and subcellular fractionation experiments are used to determine whether FLAG-mTOR(S2035T, ΔHT19) is localized to the ER/Golgi. The functional significance of mTOR localization is determined as follows. In the absence of rapamycin, FLAG-mTOR(S2035T) and endogenous mTOR are both localized to the ER and Golgi, and are both capable of promoting S6 phosphorylation. In the presence of rapamycin, FKBP12-rapamycin binds to and inhibits the endogenous mTOR, but not FLAG-mTOR(S2035T) or FLAG-mTOR(S2035T, ΔHT19). This allows specific examination of the function of FLAG-mTOR(S2035T, ΔHT19) by detecting S6 phosphorylation.

(B) Deletion of HT19 inhibits the ability of mTOR to localize to the ER. FLAG-mTOR(S2035T) and FLAG-mTOR(S2035T, ΔHT19) are transiently expressed in HeLa cells. Their localization was determined by IF staining using a FLAG antibody (Red). The ER is visualized by IF staining with a calnexin antibody (Green).

(C) Deletion of HT19 inhibits the ability of mTOR to localize to the Golgi. The same as FIG. 3B except Golgin-97 antibody was used instead (Green).

(D) Deletion of HT19 inhibits the ability of mTOR to associate with intracellular membranes. FLAG-mTOR(S2035T) and FLAG-mTOR(S2035T, ΔHT19) were transiently expressed in HEK293T cells. The cells were lysed in hypotonic buffer and the cell lysates were fractionated by differential centrifugation. The distribution of different proteins was determined by Western blot.

FIG. 5: Deletion of HT19 inhibits the ability of mTOR to promote S6 phosphorylation

(A) FLAG-mTOR(S2035T) and FLAG-mTOR(S2035T, ΔHT19) were transiently expressed in HeLa cells. These cells were then treated with the drug carrier (−Rap) or with rapamycin (+Rap) for 2 hrs, and analyzed for the expression of FLAG-mTOR variants and S6 phosphorylation by IF staining with a FLAG antibody and a phosphor-S6 (P-S6)-specific antibody, respectively. The nuclei were stained with DAPI. Arrowheads show FLAG-mTOR(S2035T)- and FLAG-mTOR(S2035T, ΔHT19)-expressing cells.

(B) Expression of FLAG-mTOR proteins does not affect S6 expression. This experiment is performed the same way as FIG. 4A except a S6-specific antibody instead of the phospho-S6 antibody was used.

(C) Deletion of HT19 does not affect mTOR kinase activity. FLAG-mTOR(S2035T), FLAG-mTOR(S2035T, ΔHT19) or FLAG-mTOR(S2035T, D2357E)(kinase-dead) were transiently expressed in HEK293T cells. After immunoprecipitation, they were assayed for mTOR kinase activity toward GST-p70S6K1(aa333-412). Thr389 phosphorylation was detected by Western blot with a P-Thr389 antibody.

FIG. 6: Overexpression of GLS-EGFP and ELS-EGFP inhibits mTORC1

(A) Overexpression of GLS-EGFP and ELS-EGFP inhibits S6 phosphorylation in HeLa cells. EGFP, ELS-EGFP and GLS-EGFP were transiently expressed in HeLa cells. S6 phosphorylation was determined by IF using a P-S6-specific antibody. The arrowheads show GFP-positive cells.

(B) Quantification of cells with P-S6 signal from the FIG. 6B experiment. The expression level of EGFP fusion proteins in individual cells was categorized into strong (S), moderate (M), and weak (W) and no (N) signal according to the GFP signals. Virtually all the cells expressing EGFP alone show strong GFP signal.

(C) Overexpression of GLS-EGFP and ELS-EGFP inhibits S6 phosphorylation in HEK293T cells. EGFP, ELS-EGFP and GLS-EGFP were transiently expressed in HEK293T cells. P-S6 signal was quantified and the level of GFP signal in individual cells was categorized as in FIG. 6B.

(D) Overexpression of GLS-EGFP and ELS-EGFP inhibits Thr389 phosphorylation of p70S6K1 in HEK293T cells. HA-p70S6K1 was transiently expressed in HEK293T cells together with ELS-EGFP, GLS-EGFP or EGFP. HA-p70S6K1 phosphorylation was assayed by Western blot using a P-Thr389-specific antibody.

(E) ELS-EGFP, GLS-EGFP or EGFP was transiently expressed in HEK293T cells. The size of EGFP-positive cells was measured by FACS.

FIG. 7: Overexpression of GLS-EGFP and ELS-EGFP inhibits mTORC2

(A) Overexpression of GLS-EGFP and ELS-EGFP disrupts normal actin organization and inhibits normal cell spreading. EGFP, GLS-EGFP and ELS-EGFP were transiently expressed in HeLa cells. Actin cytoskeleton organization was determined by phalloidin-TRITC staining. The transfected cells are GFP-positive.

(B) Overexpression of GLS-EGFP and ELS-EGFP inhibits cell spreading. The level of GFP signal in individual cells was categorized as in FIG. 6B.

(C) ELS and GLS do not inhibit AKT/PKB phosphorylation in HEK293 cells. ELS-EGFP, GLS-EGFP or EGFP was transiently expressed with GST-AKT in HEK293 cells. The cells were starved from fetal bovine serum for 4 hrs before incubated with insulin for 15 min. Phosphorylation of GST-AKT and endogenous AKT was analyzed by Western blot with a P-Ser473 antibody.

(D) ELS and GLS do not inhibit AKT/PKB phosphorylation in HEK293T cells. ELS-EGFP, GLS-EGFP or EGFP was transiently expressed with GST-AKT in HEK293T cells. Phosphorylation of GST-AKT and endogenous AKT was analyzed by Western blot with a P-Ser473 antibody.

FIG. 8:

A. GLS-GFP, ELS-GFP and GFP are expressed in HEK293 cells (human embryonic kidney cells transformed by adenovirus 5). GLS-GFP and ELS-GFP, but not GFP alone cause membrane blebbing, a phenotype associated with apoptosis.

B. TUNEL (Terminal transferase dUTP nick end labeling) shows that GLS-GFP and ELS-

GFP expressing HEK293 cells undergo high level of apoptosis.

C. Quantification of apoptotic cells from B.

FIG. 9:

ELS and GLS nucleotide sequences

### DETAILED DESCRIPTION OF THE INVENTION

The present invention is drawn to GLS and ELS polypeptides, as well as DNA segments encoding GLS and ELS polypeptides and antibodies. The present invention also provides methods of making such DNA segments and polypeptides, as well as their use as research tools, in drug screening, diagnosis of various clinical conditions associated with mTor, immunosuppressive and cancer therapy. Cells relating to GLS and ELS are disclosed as well.

Studies have shown that TOR proteins are associated with light intracellular membranes in mammals and yeast (Hara et al., 2002; Kim et al., 2002; Kim et al., 2003; Wedaman et al., 2003), likely to include the ER and the Golgi. TCS2 and Rheb, two key upstream regulators of mTOR, have been found to be localized to the ER and Golgi (Wienecke et al., 1996; Jones et al., 2004; Buerger et al., 2006). Importantly, the small GTPase Rheb directly interacts with mTOR (Long et al., 2005a; Long et al., 2005b) and the Golgi localization of Rheb is critical for its ability to stimulate mTOR activity (Buerger et al., 2006). Together with our data, these observations suggest that the ER and the Golgi are common anchors for TOR signaling complexes. The identification of ER- and Golgi-localization sequences laid an important foundation for future study of the mechanism of mTOR localization. Deletion of HT19, a common element for both ELS and GLS, causes mTOR delocalization and inhibits mTOR signaling to S6 phosphorylation. Moreover, overexpression of ELS and GLS not only inhibits S6 and S6K1 phosphorylation, but also causes disorganization of the actin stress fibers, important downstream events for mTORC1 and mTORC2, respectively, that ELS and GLS dominant-negatively interfere with the functions of both mTORC1 and mTORC2. Altogether, anchoring on the ER/Golgi is important for signaling by both mTOR complexes. The ER and the Golgi have been shown to anchor signaling by SREBPs into the nucleus (Brown et al., 2002; Yang et al., 2002; Rawson, 2003).

The ER and the Golgi are part of the secretory pathway that actively engages transport of proteins to the plasma membrane through the vesicular trafficking. Each organelle also has to maintain a unique stable set of resident proteins that define its structural and functional properties. The ER residency is typically achieved by preventing resident proteins from entering the transport vesicles, or by retrieval of those being transported to the Golgi. The first mechanism is exemplified by SREBP, whose interaction with the COPII vesicle-formation proteins Sar1, Sec23 and Sec24 is inhibited by high cholesterol concentration (Rawson, 2003). The second is directed by discrete retrieval motifs: soluble luminal proteins with the H/KDEL sequence at the carboxy-terminus, or membrane proteins have a dibasic motif (KK or RR) located close to the terminus of the cytosolic domain (Teasdale and Jackson, 1996). Recent work has identified the FFAT (diphenylalanine [FF] in an Acidic Tract) motif responsible for localizing cytosolic proteins to the cytoplasmic face of the ER (Loewen et al., 2003). The FFAT motif has the consensus amino acid sequence EFFDAxE. FFAT motifs bind to the highly conserved VAP proteins that are anchored to the cytoplasmic face of the ER (Loewen et al., 2003). Studies of several Golgi-resident proteins have revealed several mechanisms for Golgi-targeting. For example, Imh1 is recruited to the Golgi membrane through the interaction of two GRIP domains with the Arf-like small GTPase Arl1. Arl1 is recruited to the Golgi by another member of the Arl family, Arl3, which requires an amino-terminal acetylated methionine residue to bind to a Golgi-localized, integral membrane protein called Sys1 (Graham, 2004). ArfGAP1 interaction with the Golgi is mediated by interaction of a hydrophobic motif with curved membrane lipid bilayer (Bigay et al., 2003; Pamis et al., 2006).

Sequence analysis of ELS and GLS shows that they bear no apparent sequence similarity to known ER-targeting signals. Moreover, extensive homology search fails to reveal any significant similarity between ELS/GLS and known ER/Golgi surface proteins.

### WORKING EXAMPLES

Cell Lines, Plasmids, Antibodies and Other Reagents

Mammalian cell lines used in this study were purchased from American Type Culture Center (ATCC) and maintained as recommended by the supplier. To construct plasmids expressing EGFP- or FLAG-HA-mTOR fusion proteins, different mTOR regions were generated by polymerase chain reaction (PCR) and cloned into pEGFPN1 (Clontech) and pFH-IRESneo (Teichmann et al., 2000). Further deletional analysis of different regions of GLS-EGFP was carried out by the inverted PCR approach using primers flanking the deleted sequence. FLAG-mTOR(S2035T, ΔHT19) was created by deletion of HEAT19 from FLAG-mTOR(S2035T)(Brown et al., 1995) by the inverted PCR approach. The N-terminal mTOR/FRAP antibody was described and characterized previously (Drenan et al., 2004). Other antibodies were obtained from the following sources: FLAG antibodies (Sigma); Calnexin antibodies (BD Transduction Laboratories); GFP antibody (Affinity BioReagents); Golgin-97 antibody and goat anti-mouse or rabbit secondary antibodies conjugated to Alexa Fluor 594 or 488 (Molecular Probes); HA monoclonal antibodies (Harlan Laboratories); AKT, P-AKT(Ser473), S6, P-S6(Ser235/236) and P-S6K1(Thr389) antibodies (Cell Signaling); horseradish peroxidase (HRP)-conjugated goat anti-mouse or anti-rabbit antibodies (Pierce). Proteinase K and phalloidin-TRITC were purchased from Sigma.

Immunofluorescence (IF), Western blot, Immunoprecipitaiton and In Vitro Kinase Assay

For immunofluorescence, cells were fixed in 3% paraformaldehyde, 2% sucrose in H₂O for 10 min at 37° C., permeabilized with ice-cold HEPES-Triton X-100 buffer (0.5% Triton X-100 in 20 mM HEPES, pH 7.4, 50 mM NaCl, 3 mM MgCl₂, 300 mM sucrose) for 5 min on ice, blocked with 0.1% BSA in PBS for 10 min on ice, and incubated with primary antibodies for 20 min (N-terminal mTOR antibody at 1:500, mouse anti-Calnexin at 1:50, mouse anti-Golgin-97 at 1:100, rabbit anti-FLAG at 1:1000) at 37° C. in a moisture chamber. Unbound antibodies were removed by washing ten times with TRIS-buffered Saline plus 0.1% Tween 20 (TBST). Secondary antibodies labeled with Texas Red-X or Alexa Fluor 488 (Molecular Probes) were incubated for 15 min at room temperature and washed as with the primary antibodies. Glass cover slips carrying treated cells were mounted with Cytoseal mounting medium onto glass slides and analyzed using an Olympus BX51 fluorescence microscope equipped with a Qimaging Retiga EXi digital camera. Phalloidin-TRITC staining was carried according to the manufacturer's instruction (Sigma). Cell lysates for Western blot were prepared using ice-cold lysis buffer (LB) containing 50 mM HEPES-KOH (pH 7.4), 40 mM NaCl, 1 mM EDTA, 0.1% Triton X-100, 5% glycerol, 10 mM sodium pyrophosphate, 10 mM β-glycerophosphate, 1.5 mM Na₃VO₄, 50 mM NaF, and 1× protease inhibitor cocktail (Roche Diagnostics, GmbH). Protein samples were separated on SDS polyacrylamide gels and transferred onto Immobilon-P membrane. After blocking with 5% dry milk in TBST, the membrane was incubated with primary antibodies for 1 hr to overnight, followed by incubation with HRP-conjugated secondary antibodies (1:10,000) for 30 min and with ECL (Amersham Life Sciences). For kinase assays, lysates of HEK293T cells transiently transfected with FLAG-mTOR(S2035T), FLAG-mTOR(S2035T, ΔHT19) or FLAG-mTOR(S2035T, D2357E) were immunoprecipitated with the FLAG M2 antibody and protein A-Sepharose beads. After extensive wash, the immunoprecipitated materials were incubated with GST-p70S6K1 (aa333-412) that contains Thr389 and 2 mM ATP for 20 min. Phosphorylation of GST-p70S6K1 at Thr389 was detected by Western blot with a P-S6K1(Thr389)-specific antibody.

Subcellular Fractionation and Related Biochemistry

HEK293T cells were washed with ice-cold HME buffer (10 mM HEPES, 250 mM Mannitol, 0.5 mM EDTA, pH 7.4), resuspended in 5 volumes of ice-cold HME buffer containing 0.1 mM PMSF and dounce-homogenized by 10 gentle strokes. Nuclei and unbroken cells were removed at 1,500 g. The supernatants were centrifuged at 10,000 g (10 min, 4° C.). The pellets (P10) were resuspended in HME buffer. The S10 supernatant (after saving an aliquot) was overlaid on a 20% sucrose cushion and further centrifuged at 100,000 g (60 min, 4° C.). The pellets (P100) were resuspended in HME buffer. The S100 was also saved for Western blot analysis. For protease protection assays, the P100 pellets were resuspended in HME plus 10 mM CaCl₂ without or with 1% Triton X-100, and then incubated with different concentrations of trypsin/chymotrypsin (0, 5, 50 μg/ml) for 40 min at 4° C. Reactions were stopped by addition of aprotinin and boiling in SDS protein sample buffer.

HT19 is Required for Normal Subcellular Distribution and Function of mTOR

HT19 is a common element for GLS and ELS and to determine whether GLS and ELS are important for mTOR localization, HT19 from FLAG-mTOR(S2035T) was deleted. S2035 is a conserved residue located in FRB domain and is crucial for the binding of FKBP12-rapamycin (Chen et al., 1995; Zheng et al., 1995). The S2035→T mutation disrupts the binding of FKBP12-rapamycin and confers dominant rapamycin-resistant signaling for mTOR (Brown et al., 1995; Chen et al., 1995). Deletion of HT19 in this rapamycin-resistant mTOR allele also allows provides whether ER- and Golgi-localization is important for the functions of mTOR (FIG. 4A and see below). Like endogenous mTOR and FLAG-mTOR (Drenan et al., 2004), the transiently expressed FLAG-mTOR(S2035T) is localized to the ER and the Golgi, as judged by the co-localization of FLAG-mTOR(S2035T) with calnexin and Golgin-97 by IF and subcellular fractionation (FIGS. 4B and C). In contrast, IF shows that FLAG-mTOR(S2046T, HT19Δ) exhibits a highly aggregated pattern that is distinct from that of calnexin and Golgin-97 (FIGS. 4B and C). Moreover, a large proportion of FLAG-mTOR(S2046T, HT19Δ) is found in the cytosol (S100) (FIG. 4D). Together, the above results show that HT19 is important for proper mTOR localization.

The strategy for studying the functional significance of mTOR localization is illustrated in FIG. 4A. In FLAG-mTOR(S2035T)-expressing cells in the absence of rapamycin, both endogenous mTOR and FLAG-mTOR(S2035T) are localized to the ER and the Golgi, and both can send normal signal to downstream effectors such as S6, a ribosomal protein and an mTOR downstream effector. S6 phosphorylation occurs at five residues at the C-terminus (Ser235, Ser236, Ser240, Ser244, and Ser247) (Krieg et al., 1988), which is mTOR-dependent. Rapamycin treatment results in a loss of S6 phosphorylation (Jefferies et al., 1994). In the presence of rapamycin, endogenous mTOR is inhibited but FLAG-mTOR(S2035T) is not. FLAG-mTOR(S2035T) can still send normal signal to S6 phosphorylation. This assay allows us to assay for the ability of FLAG-mTOR(S2035T, ΔHT19) to signal to mTOR downstream effectors.

Transfected HeLa cells with plasmids expressing FLAG-mTOR(S2035T) or FLAG-mTOR(S2035T, HT19Δ) were treated with or without rapamycin then IF staining with a FLAG-specific mouse monoclonal antibody to identify cells expressing the FLAG-mTOR variant, and with a rabbit polyclonal antibody specific for P-S6(Ser235/236) to determine S6 phosphorylation was performed. As expected, rapamycin blocks S6 phosphorylation in non-transfected cells, but not in cells expressing FLAG-mTOR(S2035T) (FIG. 5A). In contrast, rapamycin potently inhibits S6 phosphorylation in FLAG-mTOR(S2035T, HT19Δ)-expressing cells (FIG. 5A). In the absence of rapamycin, S6 and phosphor-S6 levels are normal in cells expressing FLAG-mTOR(S2035T, HT19Δ), indicating that FLAG-mTOR(S2035T, HT19Δ) expression itself does not affect S6 phosphorylation or S6 protein levels (FIGS. 5A and B). HEAT repeats are known to fold independently (Groves et al., 1999; Perry and Kleckner, 2003). However, it is still possible that HT19 deletion indirectly disturbs an important mTOR structure such as the kinase domain and affects mTOR signaling indirectly. To explore this possibility, an assay for the kinase activity of different mTOR variants was performed. It was identified that the HT19Δ mutant, but not the D2357E kinase-dead mutant, retains the ability to phosphorylate Thr389 of S6K1 in vitro (FIG. 5C). A functional mTOR kinase toward Thr389 requires assembly of a functional mTORC1 complex that includes Raptor bound to the N-terminus and GβL associated with the C-terminus (Hara et al., 2002; Kim et al., 2002). The results indicate that mTOR maintains a relatively normal overall structure in the absence of HT19. Together, these observations suggest that ER- and Golgi-localization is crucial for normal mTOR signaling function.

Overexpression of ELS and GLS Inhibits Both mTORC1 and mTORC2

The two TOR complexes play essential roles in cell growth and function. TORC1 regulates cell growth in a rapamycin-sensitive manner, while TORC2 is rapamycin-insensitive and is involved in actin organization (Jacinto et al., 2004; Sarbassov et al., 2004) and AKT/PKB phosphorylation (Sarbassov et al., 2005). One possible mechanism for ELS and GLS localization is that they interact with an ER and Golgi resident protein(s) as in the case of SREBP1 (Rawson, 2003). Conceptually, ELS and GLS overexpression may dominant-negatively affect endogenous mTOR functions. To test this, we examined S6 phosphorylation in HeLa cells transiently expressing GLS-EGFP or ELS-EGFP. By IF with the phospho-S6-specific antibody, S6 phosphorylation was strongly inhibited in cells overexpressing GLS-EGFP or ELS-EGFP, but not in the untransfected cells or EGFP-expressing cells (FIGS. 6A and B). The degree of inhibition of S6 phosphorylation is correlative with GLS and ELS expression levels: the lowest S6 phosphorylation is found in strong (S) GLS- and ELS-expressing cells; partial inhibition of S6 phosphorylation is seen in moderate (M) and weak (W) GLS- and ELS-expressing cells; no inhibition of S6 phosphorylation in the non-transfected cells (FIG. 6B). Additionally, EGFP shows no discernible inhibition of S6 phosphorylation (FIG. 6B). Essentially the same results were obtained with HEK293T cells (FIG. 6C). To independently show that mTORC1 is inhibited, we transiently expressed HA-p70S6K1 in HEK293T cells with ELS-EGFP, GLS-EGFP or EGFP. We then assayed for HA-p70S6K1 phosphorylation by Western blot with a P-Thr389-specific antibody. Indeed, Thr389 phosphorylation is strongly inhibited by ELS-EGFP or GLS-EGFP, but not EGFP alone (FIG. 6D). Another established role of mTORC1 is cell size regulation. We found that ELS-EGFP and GLS-EGFP cells are 10-20% smaller than EGFP cells (FIG. 6E), which is consistent with the effect of rapamycin (Kim et al., 2002). Together, these observations show that overexpression of GLS and ELS strongly inhibits mTORC1 signaling.

We have also asked whether mTORC2 is affected by the overexpression of ELS and GLS. The role of mTORC2 can be tested by cell spreading and organization of actin stress fibers (Jacinto et al., 2004; Sarbassov et al., 2004). By staining cells with TRITC-conjugated phalloidin, we found that EGFP control cells or non-transfected cells spread nicely and actin stress fibers are well organized. In contrast, GLS-EGFP and ELS-EGFP-expressing cells show highly aggregated actin stress fibers. These cells are also spreaded much less (FIGS. 7A and B). The morphology of cells overexpressing ELS-EGFP and GLS-EGFP is similar to that of mTOR and mAVO3/Rictor siRNA knockdown cells (Jacinto et al., 2004; Sarbassov et al., 2004), suggesting that overexpression of ELS-EGFP and GLS-EGFP also inhibits the function of mTORC2.

### REFERENCES

Andrade, M. A., and Bork, P. (1995). HEAT repeats in the Huntington's disease protein. Nat. Genet. 11, 115-116.
Baumann, O., and Walz, B. (2001). Endoplasmic reticulum of animal cells and its organization into structural and functional domains. International Review Of Cytology 205, 149-214.
Bertram, P. G., Choi, J., Carvalho, J., Ai, W. D., Zeng, C. B., Chan, T. F., and Zheng, X. F. S. (2000). Tripartite regulation of Gln3p by TOR, Ure2p and phosphatases. J. Biol. Chem. 275, 35727-35733.
Bigay, J., Gounon, P., Robineau, S., and Antonny, B. (2003). Lipid packing sensed by ArfGAP1 couples COPI coat disassembly to membrane bilayer curvature 426, 563-566.
Bossini, R., Isacchi, A., and Sonnhammer, E. L. (2000). FAT: a novel domain in PIK-related kinases. Trends Biochem. Sci. 25, 225-227.
Brown, A., Sun, L., Feramisco, J., Brown, M., and Goldstein, J. (2002). Cholesterol addition to ER membranes alters conformation of SCAP, the SREBP escort protein that regulates cholesterol metabolism. Mol Cell 10, 237-245.
Brown, E. J., Beal, P. A., Keith, C. T., Chen, J., Shin, T. B., and Schreiber, S. L. (1995). Control of p70 s6 kinase by kinase activity of FRAP in vivo. Nature 377, 441-446.
Buerger, C., DeVries, B., and Stambolic, V. (2006). Localization of Rheb to the endomembrane is critical for its signaling function. Biochem Biophys Res Commun 344, 869-880.
Chen, J., Zheng, X. F., Brown, E. J., and Schreiber, S. L. (1995). Identification of an 11-kDa FKBP12-rapamycin-binding domain within the 289-kDa FKBP12-rapamycin-associated protein and characterization of a critical serine residue. Proc. Natl. Acad. Sci. USA 92, 4947-4951.
Chiu, V., Bivona, T., Hach, A., Sajous, J., Silletti, J., Wiener, H., Johnson, R., Cox, A., and Philips, M. (2002). Ras signalling on the endoplasmic reticulum and the Golgi. Nat. Cell Biol 4, 343-350.
Chook, Y. M., and Blobel, G. (1999). Structure of the nuclear transport complex karyopherin-beta2-Ran×GppNHp [see comments]. nature 399, 230-237.
Dennis, P. B., Fumagalli, S., and Thomas, G. (1999). Target of rapamycin (TOR): balancing the opposing forces of protein synthesis and degradation. Curr. Opin. Genet. Dev. 9, 49-54.
Desai, B. N., Myers, B. R., and Schreiber, S. L. (2002). FKBP12-rapamycin-associated protein associates with mitochondria and senses osmotic stress via mitochondrial dysfunction. Proc. Natl. Acad. Sci. USA 99, 4319-4324.
Drenan, R. M., Liu, X., Bertram, P. G., and Zheng, X. F. S. (2004). FKBP12-Rapamycin-associated Protein or Mammalian Target of Rapamycin (FRAP/mTOR) Localization in the Endoplasmic Reticulum and the Golgi Apparatus. J. Biol. Chem. 279, 772-778.
Graham, T. (2004). Membrane targeting: getting Arl to the Golgi. Curr Biol 14, R483-485.
Groves, M. R., Hanlon, N., Turowski, P., Hemmings, B. A., and Barford, D. (1999). The structure of the protein phosphatase 2A PR65/A subunit reveals the conformation of its 15 tandemly repeated HEAT motifs. Cell 96, 99-110.
Hara, K., Maruki, Y., Long, X., Yoshino, K., Oshiro, N., Hidayat, S., Tokunaga, C., Avruch, J., and Yonezawa, K. (2002). Raptor, a binding partner of target of rapamycin (TOR), mediates TOR action. Cell 110, 177-189.
Huang, S., and Houghton, P. (2003). Targeting mTOR signaling for cancer therapy. Curr Opin Pharmacol 3, 371-377.
Hunter, T. (1995). When a lipid kinase is not a lipid kinase? When a lipid kinase is a protein kinase? Cell 83, 1-4.
Jacinto, E., Facchinetti, V., Liu, D., Soto, N., Wei, S., Jung, S., Huang, Q., Qin, J., and Su, B. (2006). SIN1/MIP1 Maintains rictor-mTOR Complex Integrity and Regulates Akt Phosphorylation and Substrate Specificity. Cell 127, 125-137.
Jacinto, E., Loewith, R., Schmidt, A., Lin, S., Ruegg, M., Hall, A., and Hall, M. (2004). Mammalian TOR complex 2 controls the actin cytoskeleton and is rapamycin insensitive. Nat Cell Biol 6, 1122-1128.
Jefferies, H. B., Reinhard, C., Kozma, S. C., and Thomas, G. (1994). Rapamycin selectively represses translation of the “polypyrimidine tract” mRNA family. Proc. Natl. Acad. Sci. USA 91, 4441-4445.
Jones, K. A., Jiang, X., Yamamoto, Y., and Yeung, R. S. (2004). Tuberin is a component of lipid rafts and mediates caveolin-1 localization: role of TSC2 in post-Golgi transport. Exp Cell Res 295, 512-524.
Keith, C. T., and Schreiber, S. L. (1995). PIK-related kinases: DNA repair, recombination, and cell cycle checkpoints. Science 270, 50-51.
Kim, D., Sarbassov, d.D., Ali, S., King, J., Latek, R., Erdjument-Bromage, H., Tempst, P., and Sabatini, D. (2002). mTOR interacts with raptor to form a nutrient-sensitive complex that signals to the cell growth machinery. Cell 110, 163-175.
Kim, d.H., Sarbassov, d.D., Ali, S., Latek, R., Guntur, K., Erdjument-Bromage, H., Tempst, P., and Sabatini, D. (2003). GbetaL, a Positive Regulator of the Rapamycin-Sensitive Pathway Required for the Nutrient-Sensitive Interaction between Raptor and mTOR. Mol. Cell. 11, 895-904.
Kim, J. E., and Chen, J. (2000). Cytoplasmic-nuclear shuttling of FKBP12-rapamycin-associated protein is involved in rapamycin-sensitive signaling and translation initiation. Proc. Natl. Acad. Sci. USA 97, 14340-14345.
Krieg, J., Hofsteenge, J., and Thomas, G. (1988). Identification of the 40 S ribosomal protein S6 phosphorylation sites induced by cycloheximide [published erratum appears in J Biol Chem 1988 Nov. 25; 263(33):17887]. J. Biol. Chem. 263, 11473-11477.
Kuruvilla, F., and Schreiber, S. L. (1999). The PIK-related kinases intercept conventional signaling pathways. Chemistry and Biology 6, R129-R136.
Li, H., Tsang, C. K., Watkins, M., Bertram, P. G., and Zheng, X. F. S. (2006). Nutrient regulates Tor1 nuclear localization and association with rDNA promoter 442, 1058-1061.
Loewen, C. J. R., Roy, A., and Levine, T. P. (2003). A conserved ER targeting motif in three families of lipid binding proteins and in Opi1p binds VAP. EMBO Journal 22, 2025-2035.
Loewith, R., Jacinto, E., Wullschleger, S., Lorberg, A., Crespo, J., Bonenfant, D., Oppliger, W., Jenoe, P., and Hall, M. N. (2002). Two TOR Complexes, Only One of which Is Rapamycin Sensitive, Have Distinct Roles in Cell Growth Control. Mol. Cell. 10, 457-468.
Long, X., Lin, Y., Ortiz-Vega, S., Yonezawa, K., and Avruch, J. (2005a). Rheb binds and regulates the mTOR kinase. Curr Biol 15, 702-713.
Long, X., Ortiz-Vega, S., Lin, Y., and Avruch, J. (2005b). Rheb Binding to Mammalian Target of Rapamycin (mTOR) Is Regulated by Amino Acid Sufficiency. J. Biol. Chem. 280, 23433-23436.
Lorenz, M. C., and Heitman, J. (1995). TOR mutations confer rapamycin resistance by preventing interaction with FKBP12-rapamycin. J. Biol. Chem. 270, 27531-27537.
McDaniel, M. L., Marshall, C. A., Pappan, K. L., and Kwon, G. (2002). Metabolic and Autocrine Regulation of the Mammalian Target of Rapamycin by Pancreatic {beta}-Cells Diabetes 51, 2877-7438.
Pahl, H. L. (1999). Signal Transduction From the Endoplasmic Reticulum to the Cell Nucleus. Physiol. Rev. 79, 683-701.
Parnis, A., Rawet, M., Regev, L., Barkan, B., Rotman, M., Gaitner, M., and Cassel, D. (2006). Golgi Localization Determinants in ArfGAP1 and in New Tissue-specific ArfGAP1 Isoforms10.1074/jbc.M508959200. J. Biol. Chem. 281, 3785-3792.
Perry, J., and Kleckner, N. (2003). The ATRs, ATMs, and TORs Are Giant HEAT Repeat Proteins. Cell 112, 151-155.
Raught, B., Gingras, A.-C., and Sonenberg, N. (2001). The target of rapamycin (TOR) protein. Proc. Natl. Acad. Sci. USA 98, 7037-7044.
Rawson, R. B. (2003). THE SREBP PATHWAY—INSIGHTS FROM INSIGS AND INSECTS. Nature Reviews Molecular Cell Biology
Nat Rev Mol Cell Biol 4, 631-640.
Rohde, J., Heitman, J., and Cardenas, M. (2001). The TOR kinases link nutrient sensing to cell growth. J. Biol. Chem. 276, 7027-7036.
Sakai, J., Duncan, E., Rawson, R., Hua, X., Brown, M., and Goldstein, J. (1996). Sterol-regulated release of SREBP-2 from cell membranes requires two sequential cleavages, one within a transmembrane segment. Cell 85, 1037-1046.
Sarbassov, D., Ali, S., Kim, D., Guertin, D., Latek, R., Erdjument-Bromage, H., Tempst, P., and Sabatini, D. (2004). Rictor, a novel binding partner of mTOR, defines a rapamycin-insensitive and raptor-independent pathway that regulates the cytoskeleton. Curr Biol 14, 1296-1302.
Sarbassov, D. D., Ali, S. M., Sengupta, S., Sheen, J.-H., Hsu, P. P., Bagley, A. F., Markhard, A. L., and Sabatini, D. M. (2006). Prolonged Rapamycin Treatment Inhibits mTORC2 Assembly and Akt/PKB. Molecular Cell 22, 159-168.
Sarbassov, D. D., Guertin, D. A., Ali, S. M., and Sabatini, D. M. (2005). Phosphorylation and Regulation of Akt/PKB by the Rictor-mTOR Complex. Science 307, 1098-1101.
Schmelzle, T., and Hall, M. N. (2000). TOR, a Central Controller of Cell Growth. Cell 103, 253-262.
Shiota, C., Woo, J.-T., Lindner, J., Shelton, K. D., and Magnuson, M. A. (2006). Multiallelic Disruption of the rictor Gene in Mice Reveals that mTOR Complex 2 Is Essential for Fetal Growth and Viability. Developmental Cell 11, 583-589.
Stan, R., McLaughlin, M. M., Cafferkey, R., Johnson, R. K., Rosenberg, M., and Livi, G. P. (1994). Interaction between FKBP12-rapamycin and TOR involves a conserved serine residue. J. Biol. Chem. 269, 32027-32030.
Teasdale, R. D., and Jackson, M. R. (1996). SIGNAL-MEDIATED SORTING OF MEMBRANE PROTEINS BETWEEN THE ENDOPLASMIC RETICULUM AND THE GOLGI APPARATUS. Annual Review of Cell and Developmental Biology 12, 27-54.
Teichmann, M., Wang, Z., and Roeder, R. G. (2000). A stable complex of a novel transcription factor IIB-related factor, human TFIIIB50, and associated proteins mediate selective transcription by RNA polymerase III of genes with upstream promoter elements. PNAS 97, 14200-14205.
Vetter, I. R., Arndt, A., Kutay, U., Gorlich, D., and Wittinghofer, A. (1999). Structural view of the Ran-Importin beta interaction at 2.3 A resolution. cell 97, 635-646.
Wang, X., Sato, R., Brown, M., Hua, X., and Goldstein, J. (1994). SREBP-1, a membrane-bound transcription factor released by sterol-regulated proteolysis. Cell 77, 53-62.
Wedaman, K. P., Reinke, A., Anderson, S., Yates, J., III, McCaffery, J. M., and Powers, T. (2003). Tor Kinases Are in Distinct Membrane-associated Protein Complexes in Saccharomyces cerevisiae. Mol. Biol. Cell 14, 1204-1220.
Wienecke, R., Maize, J. C., Jr, Shoarinejad, F., Vass, W. C., Reed, J., Bonifacino, J. S., Resau, J. H., de Gunzburg, J., and Yeung et, a. (1996). Co-localization of the TSC2 product tuberin with its target Rap1 in the Golgi apparatus. Oncogene 13, 913-923.
Yang, Q., Inoki, K., Ikenoue, T., and Guan, K.-L. (2006). Identification of Sin1 as an essential TORC2 component required for complex formation and kinase activity 10.1101/gad.1461206. Genes &amp; Dev. 20, 2820-2832.
Yang, T., Espenshade, P., Wright, M., Yabe, D., Gong, Y., Aebersold, R., Goldstein, J., and Brown, M. (2002). Crucial step in cholesterol homeostasis: sterols promote binding of SCAP to INSIG-1, a membrane protein that facilitates retention of SREBPs in ER. Cell 110, 489-500.
Zhang, X., Shu, L., Hosoi, H., Murti, K. G., and Houghton, P. J. (2002). Predominant Nuclear Localization of Mammalian Target of Rapamycin in Normal and Malignant Cells in Culture. J. Biol. Chem. 277, 28127-23381.
Zheng, X. F., Florentino, D., Chen, J., Crabtree, G. R., and Schreiber, S. L. (1995). TOR kinase domains are required for two distinct functions, only one of which is inhibited by rapamycin. Cell 82, 121-130.

What is claimed is:

1. A composition comprising an isolated nucleic acid comprising a sequence selected from the group set forth in the Sequence Listing as SEQ ID NO: 1 and SEQ ID NO: 2.


SEQUENCE LISTING (2 sequences)

SEQ ID NO: 1 - DNA, 240 bp, Homo sapiens
gagttctacccagctgtgtccatggtggccctgatgcggatcttccgagaccagtcactctctcatcatcacaccatggttgtccaggccatcaccttcatcttcaagtccctgggactcaaatgtgtgcagttcctgccccaggtcatgcccacgttccttaatgtcattcgagtctgtgatggggccatccgggaatttttgttccagcagctgggaatgttggtgtcctttgtgaag

SEQ ID NO: 2 - DNA, 282 bp, Homo sapiens
actagtgaaatgctggtcaacatgggaaacttgcctctggatgagttctacccagctgtgtccatggtggccctgatgcggatcttccgagaccagtcactctctcatcatcacaccatggttgtccaggccatcaccttcatcttcaagtccctgggactcaaatgtgtgcagttcctgccccaggtcatgcccacgttccttaatgtcattcgagtctgtgatggggccatccgggaatttttgttccagcagctgggaatgttggtgtcctttgtgaag